(19) United States
(12) Patent Application Publication
(10) Pub. No.: US 2008/0287885 A1
(43) Pub. Date: Nov. 20, 2008

(54) Cylinder/Piston Unit Having a Non-Cylindrical Chamber

(76) Inventors: Hans-Rainer Hoffmann, Neuwied (DE); Bodo Asmussen, Bendorf (DE); Uwe Wortmann, Marburg (DE)

Correspondence Address: FROMMER LAWRENCE & HAUG, 745 FIFTH AVENUE- 10TH FL., NEW YORK, NY, 10151, US

(21) Appl. No.: 12/093,537

(22) PCT Filed: Nov. 2, 2006
(86) PCT No.: PCT/EP2006/010506

§ 371 Date: May 13, 2008

(30) Foreign Application Priority Data
Nov. 14, 2005 (DE) 10 2005 054 600.5

(51) Int. Cl. A61M 5/315; A61M 5/31

(52) U.S. Cl. 604/222

(57) ABSTRACT

The invention relates to a cylinder/piston unit having a cylinder and a piston which is guided therein, wherein the cylinder and the piston enclose a chamber which can be filled at least for a time with an additive, and the cylinder has at least one exit element at its front end. Here, the cross section of the chamber or the cross section of the cylinder inner wall is increased at least in regions of the cylinder from the front to the back, wherein the cylinder end having the exit element is at the front. The piston has an elastic skirt at least in the front region, the front outer edge of said elastic skirt defining a cross-sectional area in the case of an unloaded piston, which is greater than an area which is defined by a contour line. The present invention develops a cylinder/piston unit which ensures simple and reliable handling and can be stored for a long time in the filled state, in a manner which is impervious to gas and moisture.

The invention relates to a cylinder/piston unit with a cylinder and with a piston guided therein, the cylinder and the piston enclosing a chamber that can be filled at least temporarily with active substance, and the cylinder having at least one discharge element at its front end.

An ampoule for a needleless injection device is known from DE 201 05 183 U1. Located in the ampoule, inside a cylindrical chamber, there is a medicament which, for subcutaneous administration, is ejected as a jet of liquid by means of a cylindrical piston. The piston of the commercially available product is lubricated by means of silicone gel in the cylindrical chamber. When these ampoules are used in a conventional injection device, the ejection pressure drops considerably over the piston stroke. Moreover, the silicone-containing lubricant for the piston is discharged with each dose of medicament.

The object of the present invention is therefore to develop a cylinder/piston unit which, while having a small overall volume and requiring few component parts, ensures simple and safe handling and, in the filled state, is closed off in a manner impervious to gas and moisture and can be stored over long periods.

This object is achieved by the features of the main claim. The cross section of the chamber in the cylinder or the cross section of the inner wall of the cylinder increases at least in some areas from the front towards the rear, the cylinder end with the discharge element being at the front. The piston comprises, at least in the front area, an elastic skirt whose front outer edge, when the piston is unloaded, covers a cross-sectional surface area that is greater than a surface area covered by a contour line.

By means of the invention, a cylinder/piston unit is created which can be used, for example, in a subcutaneous injection device and in which, as a result of the structural configuration of the inner wall of the cylinder and of the outer contour of the piston, the drop in pressure at the discharge element over the piston stroke is much less than in known cylinder/piston units that are operated in the same way. Moreover, the cylinder/piston unit comprises a piston which is self-sealing, in accordance with the technical principle of self help, and which, by virtue of the configuration of its sealing means, sits in the cylinder free of lubricant.

Further details of the invention will become clear from the dependent claims and from the following description of illustrative embodiments depicted schematically in the figures, where:

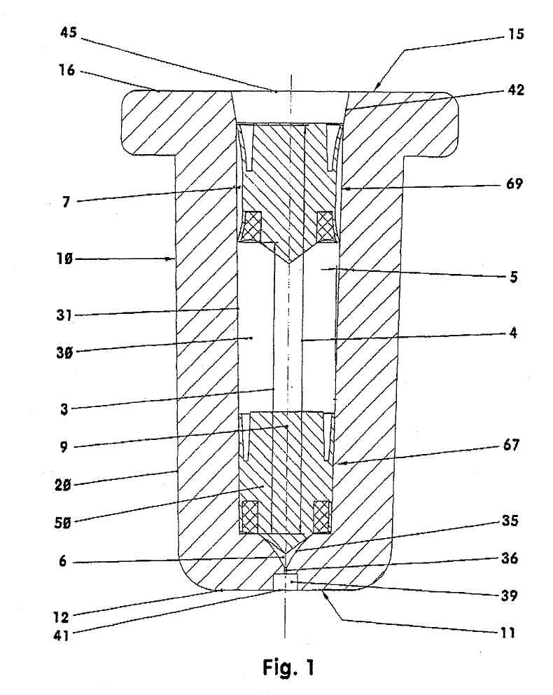

FIG. 1 shows a cylinder/piston unit, with a piston at two end positions;

FIG. 2 shows a cylinder/piston unit with the end faces closed off by films;

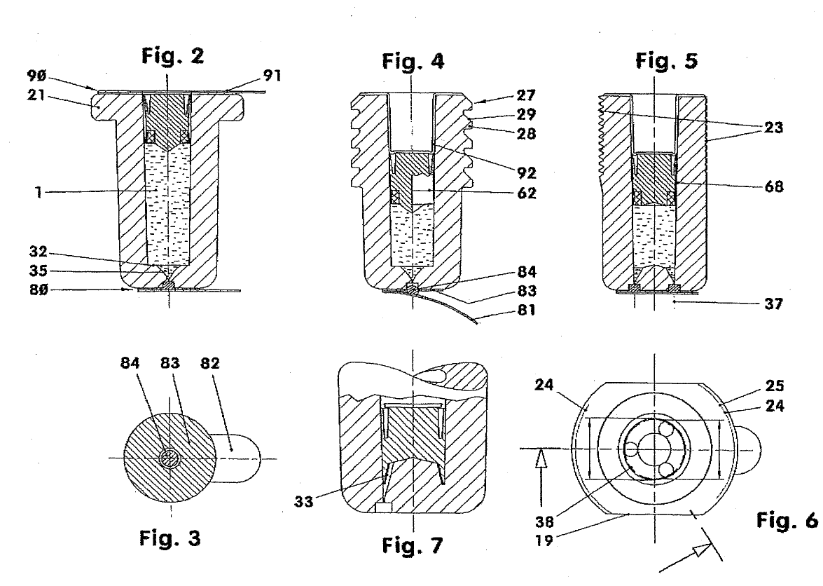

FIG. 3 shows a plan view (enlarged by 50%) of the front closure film coated with adhesive;

FIG. 4 shows a cylinder/piston unit with the closure film partially detached;

FIG. 5 shows a cylinder/piston unit with several nozzles;

FIG. 6 shows a plan view (enlarged by 50%) of the cylinder from FIG. 5;

FIG. 7 shows partial section through an emptied cylinder/piston unit with piston, and without separate sealing element;

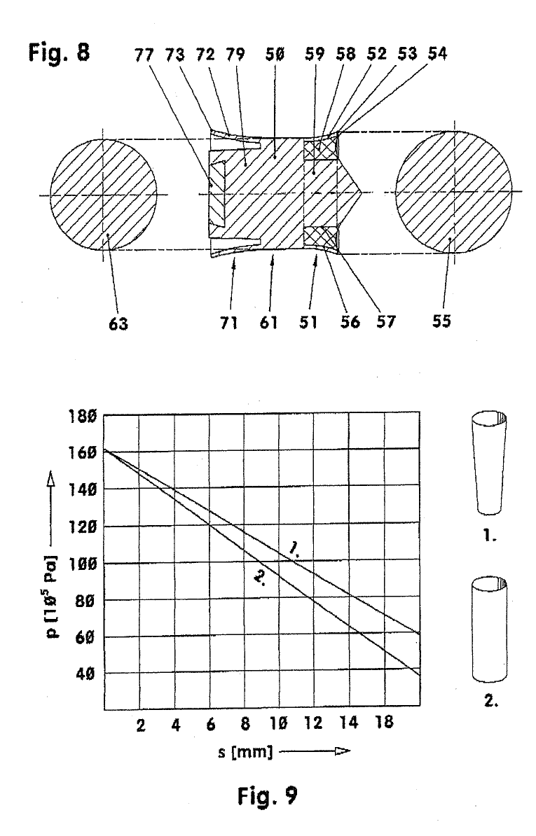

FIG. 8 shows cross sections of the unloaded piston;

FIG. 9 is a diagram of pressure and piston stroke.

FIG. 1 shows a cylinder/piston unit as is used, for example, in a subcutaneous injection device. It comprises a cylinder (10) and a piston (50), for example without a piston rod. Both enclose, within a chamber (30), a product (1) that is to be administered subcutaneously or a liquid carrier material, for example distilled water or physiological saline solution (see FIGS. 2, 4 and 5). For better clarity, the piston (50) in FIG. 1 is shown in a front position (67) and in a rear position (69). The cylinder/piston unit is, for example, designed to be used once and then disposed of. It is used to administer a volume of medicament of 0.1 to 2 ml, for example. If appropriate, a volume of medicament of 3 ml can also be administered. The cylinder (10), designed here only by way of example without an integrated injection needle, withstands a temporary pressure load of at least 300×10⁵ Pa during use in a subcutaneous injection device.

The cylinder (10) has roughly the shape of the syringe barrel of a standard disposable syringe. At the front end (11), there is a nozzle-like discharge element (36) which, in the front and, for example, flat end face (12) of the cylinder, terminates in what is for example a circular opening (41) of a free jet aperture (39). If appropriate, instead of the nozzle-like discharge element, an injection needle (not shown in the present figures) can be fitted.

An adapter flange (21), a flange (27) with locking ribs (see FIG. 4), a threaded flange (23) (see FIG. 5), a bayonet-type flange or something comparable to these is integrally formed on or secured on the rear end. Here too, the rear end face (16) of the cylinder in the area of the flange can be flat and perpendicular to the centre line (9) of the cylinder.

Situated between the adapter (21, 23, 27) and the front end face (12), there is an outer contour (20) with, for example, a cylinder jacket shape or a frustoconical shape. The shape of the outer contour (20) of the cylinder (10) is in most cases independent of the functional designation “cylinder (10)”. The outer contour (20) can, among other things, have one or more partial flattened areas in order to avoid its inadvertently rolling to the sides when handled on a flat support surface.

The adapter flange (21) according to FIGS. 1 and 2 is used, like the other adapter contours (23, 27), to fix the cylinder in a dimensionally stable and partially height-variable manner on the subcutaneous injection device. Here, a collar of the injector housing or another adapter contour engages round the corresponding flange of the cylinder (10). An adapter can be dispensed with in the case of an injector design having an almost complete cylinder holder on the injector housing.

The external diameter of the adapter flange is, for example, greater by at least one cylinder wall thickness than the external diameter of the adjacent outer contour (20) of the cylinder (10). The flange thickness is of the order of the thickness of the cylinder wall. The flange too can have one or more flattened areas (19) about its sides in order to avoid a rolling movement (see FIGS. 5 and 6). Instead of the flattened areas (19), it is also conceivable to provide notches, grooves, beads or flutings.

In FIG. 2, a cylinder (10) is shown that has a flange (27) with locking ribs. The locking ribs (28) form, in cross section, a kind of sawtooth profile with five teeth and four interstices between these. By means of the rearwardly oriented 450 bevels (29) of the teeth, the cylinder (10) can be inserted into the injector housing in, for example, five different locking positions. A corresponding housing mantle engages, for example elastically, in the corresponding annular space of the tooth interstices.

The thread (25) of the threaded flange (23) according to FIGS. 5 and 6 covers, relative to the circumference, ca. 60% of the flange contour in two threaded portions (24), for example lying opposite one another.

In the illustrative embodiments shown, the flange (27) with locking ribs and the threaded flange (23) extend along the rear 50% of the length of the cylinder.

In the case of a cylinder with only one discharge element (36), the inner contour of the cylinder (10) comprises the cylinder inner wall (31), if appropriate with a bevel (42), a cylinder base (32), a discharge funnel (35), a nozzle bore (36), and a free-jet aperture (39).

According to the illustrative embodiments shown, the cylinder inner wall (31), which is smooth for example, tapers linearly from the rear forwards. According to FIGS. 1, 2, 4 and 5, it also extends over the entire piston stroke area (4). All cross sections of the inner wall (31) of the cylinder outside the area of the discharge funnel or funnels (36) are also circular. For example, the cylinder inner wall (31) only narrows over a piston stroke (3) of 18 millimetres from a diameter of 7 millimetres to 6 millimetres. This corresponds to a taper angle of about 3.2 degrees.

Instead of the specific cases shown here, the cross sections can also change their shape, in addition to their surface area, over the piston stroke (3). Thus, the cylinder inner wall could for example have an oval cross-sectional shape at its rear end, while a cross section lying near the front end has a round or polygonal shape. Moreover, it is also possible for the change in cross-sectional shape along the piston stroke to be non-linear. For example, in order to reduce the piston braking action, the taper can start only in the final third of the ejection stroke. The transition between portions having different cross sections is generally constant.

Between the inner wall (31) of the cylinder and the rear end face (16), a 15° bevel (42) can be provided in order to make fitting of the piston (10) easier.

The cross-sectional taper can, if appropriate, also relate only to the chamber (30). In this case, the piston (50) arranged in a rear position (69) is situated along its entire length in a wall portion with, for example, a cylindrical contour.

The discharge funnel (35) tapers between the cylinder base (32) and the nozzle bore (36) in a non-linear manner, in order to permit better flow guidance. A constant transition between the discharge funnel (35) and the nozzle bore (36) is sought. The nozzle bore (36), whose diameter lies for example between 0.1 and 0.2 millimetres, is two to four times as long as its diameter. The nozzle bore (36) is adjoined by a free-jet aperture (39) in the shape of a cylinder chamber. The aperture (39) has a flat base, which is additionally oriented perpendicular to the centre line of the nozzle bore (36). Its diameter corresponds to eight to sixteen times the nozzle bore diameter, if the aperture depth is at least twice as great as the nozzle bore length.

FIGS. 5 and 6 show, inter alia, a cylinder (10) with three discharge elements in the form of nozzle bores (36). The nozzle bores (36) have centre lines (37) that are parallel to the centre line (9). They are arranged in an equidistant formation on a hole circle (38). The latter is only slightly smaller than the minimum chamber diameter in the piston stroke area (4). Oblique funnels extend between the respective nozzle bore (36) and the cylinder base (32). The cylinder base (32) bulges inwards between the funnels.

The material used for the cylinder (10) is a transparent, amorphous thermoplastic, for example a copolymer or copolymers based on cycloolefins and ethylenes or α-olefins (COC).

The piston (50) guided in the cylinder (10) must compensate for the change in cross section of the cylinder inner wall by having a corresponding reduction in its sealing cross section. The wall friction should be allowed to increase only to an inappreciable extent.

To achieve this inter alia, the piston (50) is divisible into three portions (51, 61, 71) and has, in a front portion (51) and rear portion (71), in each case a skirt (52, 72), see FIG. 8. The central piston portion (61) is located between the portions (51) and (71).

The central portion (61) has the shape of a truncated cone. It fits into the front end of the chamber (30) in a manner free from deformation. At the front, it is adjoined centrally by a front core (59). The front skirt (52) is situated around the core (59). According to FIG. 8, an axial annular groove (57) lies between the skirt (52) and the core (59). The rear skirt (72) and the rear core (79) also have a comparable structure. The skirts (52, 72), the cores (59, 79) and the central portion (61) each have a rotationally symmetrical basic shape. All the parts and structural components mentioned have congruent centre lines. The individual core (59, 79) protrudes past the respective skirt (52, 72) by a few tenths of a millimetre, for example.

According to FIGS. 8, 1, 2 and 4, the front core (59) has a straight, positive cone envelope as its end face. According to FIGS. 5 and 7, the cone envelope of the front end face is negative, that is to say shaped inward towards the centre of gravity of the piston. Almost any other rotationally symmetrical end face is conceivable, as long as it ensures that, with the piston (50) lying in the front position (67), it leaves the least possible residual volume (6) relative to the cylinder base (32) lying at least partially on it.

The front skirt (52), which extends along a quarter to a third of the piston length, is a thin-walled ring that opens in a funnel shape in the unloaded state. The front outer edge (53) of the skirt (52) encloses a cross-sectional surface area (55) which, according to FIG. 8, is greater than a cross-sectional surface area (63) whose circumference is defined by an imaginary contour line (62), lying at the foot of the skirt (52). The contour line (62) is indicated by broken lines in a partial view of the piston (10) in FIG. 4.

During a working stroke, the contour line (62) does not change its length or only barely changes its length, i.e. the cross section (63) enclosed by it remains essentially constant. By contrast, with linear tapering of the inner wall (31) of the cylinder, the front outer edge (53) shortens over the entire working stroke. In the front piston stroke area (4) (see FIG. 1), the front outer edge (53) is even shorter than the contour line (62) in the area of the sealing element (58).

According to FIGS. 1, 2, 4, 5 and 8, the sealing element (58) is located in the axial annular groove (57). The sealing element (58) is a separate sealing ring or an inserted permanently elastic sealing compound. When the piston (50) has arrived in the front position (67), said sealing element (58) connects the front inner edge (54) of the skirt (52) flush with the front end face towards the core. This contributes to minimizing the residual volume (6) in the chamber (30).

The sealing element (58) can also extend inside the skirt (52), that is to say can completely replace the front core (59). In both cases, the sealing element (58) bears sealingly on the inner wall (56) of the skirt. The pressure forces that arise during the working stroke act indirectly on the inner wall (56) of the skirt via the sealing element (58).

Moreover, it is possible to dispense with the sealing element (58) (see FIG. 7). There, the front skirt (52) protrudes into a corresponding annular groove (33).

According to FIG. 8, a magnetic or magnetizable metal plate (77) is arranged in the rear core (79) of the piston (50). It covers, for example, 50% of the rear cross-sectional surface area and is 0.5 to 1 millimetre thick. The metal plate (77) facilitates the handling of the piston (50) upon automatic assembly of the cylinder/piston unit. By means of the magnetic force and/or gravitational force of the metal plate (77), the piston (50) can be oriented and received in a targeted manner.

A tetrafluoroethylene/hexafluoropropylene copolymer (FEP) is used as the material for the piston (50). This material has self-lubricating properties in conjunction with the aforementioned material of the cylinder (10), so that no separate lubricating agents are needed between piston (50) and cylinder (10). Alternative materials that can be chosen are, among others, perfluoroalkoxy copolymer (PFA), tetrafluoroethylene (TFE) or polyvinylidene fluoride (PVDF).

If appropriate, it is also possible to use a combination of materials in which the core area (59, 61, 79) of the piston (50) is made from a material of low elasticity, while the skirts (52, 72) are made from a highly elastic material.

According to FIG. 1, the piston (50), in its rear position (69), bears resiliently on the inner wall (31) of the cylinder via the skirts (52, 72). Since the internal diameter is relatively large in this area of the cylinder, a gas-filled or air-filled gas cushion (7) forms between the radial outer wall of the piston and the inner wall (31) of the cylinder. If the piston (50) is now actuated by a corresponding drive mechanism of the subcutaneous injector, the cylinder's inner wall (31) narrows over the stroke and causes the compacting gas cushion (7) to be displaced counter to the direction of movement of the piston. The gas escapes at overpressure continuously from between the rear outer edge (73) of the skirt (72) and the inner wall (31) of the cylinder. In doing so, the rear skirt (72) lifts from the inner wall of the cylinder by an amount in the μm range. With the lubrication provided by the gas, the advancing skirt (72) slides almost free from friction along the inner wall (31) of the cylinder. Only in the lower position (67) of the piston is the gas cushion (7) almost completely displaced. By contrast, the front skirt (52) bears with a sealing action, at least via the front outer edge (53), permanently on the inner wall (31) of the cylinder.

During the working stroke of the piston (50), the liquid (1) with which the cylinder is filled is discharged through the nozzle bore (36) in a hard jet of liquid. If, for example, a mechanical, pneumatic or comparable kind of spring, or a system of springs, is used for the drive mechanism, then the drive force generally subsides continuously over the piston stroke. Consequently, the pressure of the jet of liquid also subsides accordingly. As a result of the narrowing of the cross section of the inner wall of the cylinder over the piston stroke (3), the effective piston surface becomes increasingly smaller. By this means, the pressure of the jet of liquid reduces considerably less than in the case of a cylinder with a cylindrical inner wall (see FIG. 9).

In FIG. 9, these relationships are depicted in a diagram of pressure over travel. The pressure (p) is plotted in pascals on the abscissa. The piston stroke (s) is plotted in millimetres on the ordinates. The curve (1.) shows the pressure profile in a conical chamber (30) according to FIG. 1, while the curve (2.) shows the pressure profile for a cylindrical chamber. The curve (1.) is flatter than the curve (2.). This means that a higher pressure is available to the jet of liquid shortly after the start of the jet and until the content (1) has been used up, and the difference in the pressures, dependent on travel, increases permanently as the piston stroke increases.

In a cylinder/piston unit, the two end faces (12, 16) of the cylinder (10) can have openings (41, 45) closed off by closure means (80, 90) that are impervious to gas and moisture. These closure means (80, 90) are films (81, 91) and/or coatings (92).

Filled cylinder/piston units are shown in FIGS. 2, 4 and 5. According to FIG. 2, the rear end face (16) of the cylinder (10) is closed by a closure means (90) consisting of a detachable sealing film (91) that is impervious to gas and liquid. The siliconized sealing film (91) is, for example, a PET film, an HTPE film, a PE film or a BOPP film that is bonded or sealed onto the end face (16) of the cylinder.

In FIGS. 4 and 5, a spray-on coating (92) is used instead of a sealing film (91). The sprayed-on lacquer (92) is based on a cellulose derivative. It can also be made from a comparable and biocompatible material. The sprayed-on lacquer (92) is applied sealingly to the rear end face (16), to part of the cylinder inner wall (31) and to the rear end face of the piston (50). When using the cylinder/piston unit sealed in this way, the lacquer (92) does not have to be removed before insertion into the injector. It is simply torn open by the injector ram driven by the piston (50) (see FIG. 7). In the latter figure, a residue of the lacquer (92) can be seen adhering to the piston (50).

The opening/openings (41) on the front end face (12) of the cylinder is/are closed off by a detachable sealing film (81) that comprises at least two different adhesive regions, the first adhesive region, arranged around the opening/openings (41), consisting of a contact adhesive (83) which has a greater affinity to the end face (12) of the cylinder than to the sealing film (81), while the second adhesive region, covering the opening/openings (41), contains a closure adhesive (84) that has a greater affinity to the sealing film (81) than to the material of the cylinder.

### LIST OF REFERENCE NUMBERS

1 active substance, filling
3 piston stroke
4 piston stroke area
5 half taper angle
6 residual volume
7 gas cushion
9 centre line
10 cylinder
11 front end, end with discharge element
12 end face, front
15 rear end
16 end face, rear
19 flattened area
20 outer contour
21 adapter flange
23 threaded flange
24 threaded portions
25 thread
27 flange with locking ribs
28 locking ribs
29 bevels
30 chamber
31 cylinder inner wall, inner contour
32 cylinder base
33 annular groove
35 outflow funnel
36 nozzle bore, discharge element
37 centre lines of nozzle bores
38 hole circle, cylinder on which centre lines (37) lie
39 free jet aperture
41 opening, front
42 chamber bevel, rear
45 opening, rear
50 piston
51 piston portion, front
52 skirt, front, elastic
53 skirt outer edge, front
54 skirt inner edge, front
55 cross section to outer edge
56 skirt inner wall
57 axial annular groove
58 piston seal, sealing ring, sealing compound
59 piston core, front
61 piston portion, central, frustoconical
62 contour line, imaginary
63 cross section to contour line (62)
67 piston position, front, forward end position
68 piston position, centre
69 piston position, rear
71 piston portion, rear
72 skirt, rear, elastic
73 outer edge, rear
77 plate, magnetizable
79 piston core, rear
80 front closure means
81 sealing film, detachable
82 tear-off tab
83 contact adhesive
84 closure with silicone adhesive
90 rear closure means
91 sealing film
92 coating

1-9. (canceled)

10. A cylinder/piston unit with a cylinder and a piston guided therein, wherein
the cylinder and the piston encloses a chamber that can be filled at temporarily with an active substance, wherein the cylinder has at least one discharge element at the front end of the cylinder,
the cross section of the chamber or the cross section of the cylinder inner wall increases at least in some areas from the front towards the rear,
the piston comprises, at least in the front area directed towards the discharge element, a front, elastic skirt whose front outer edge, when the piston is unloaded, covers a cross-sectional surface area that is greater than a surface area covered by a contour line lying in the area of the transition from the skirt to the piston portion supporting the skirt,
the skirt has a skirt inner wall on which an elastic sealing element bears, and
the sealing element, at least in the front end position of the piston, is flush with the front, inner skirt edge.

11. The cylinder/piston unit according to claim 10, characterized in that, in each stroke position of the piston, the front outer edge of the front skirt covers a cross-sectional surface area corresponding to the cross-sectional area of the cylinder inner wall covered by the contact line of the outside edge.

12. The cylinder/piston unit according to claim 10, characterized in that the cross sections of the inner wall of the cylinder in the piston stroke area are circular surfaces.

13. The cylinder/piston unit according to claim 10, characterized in that the surfaces of the cross sections of the inner wall of the cylinder change linearly at least over the piston stroke.

14. The cylinder/piston unit according to claim 10, characterized in that an axially extending annular groove is located between the front skirt and the piston core.

15. The cylinder/piston unit according to claim 14, characterized in that at least the front end position of the piston, the sealing element arranged in the annular groove is flush with the front, inner skirt edge and the end face of the piston.

16. The cylinder/piston unit according to claim 10, characterized in that the length of the front skirt is at least 25% of the piston length.

17. The cylinder/piston unit according to claim 10, characterized in that the rear portion of the piston has a rear skirt.

18. The cylinder/piston unit according to claim 10, with several openings in the front end of the cylinder, characterized in that the center lines of the discharge element lie on a cylinder whose diameter is smaller, by a nozzle bore diameter, than the mean diameter of the frontmost chamber cross section located in the piston stroke area.

19. The cylinder/piston unit according to claim 15, with several openings in the front end of the cylinder, characterized in that the center lines of the discharge element lie on a cylinder whose diameter is smaller, by a nozzle bore diameter, than the mean diameter of the frontmost chamber cross section located in the piston stroke area.

20. The cylinder/piston unit according to claim 19, characterized in that the length of the front skirt is at least 25% of the piston length.
